(19) United States
(12) Patent Application Publication
(10) Pub. No.: US 2011/0029055 A1
(43) Pub. Date: Feb. 3, 2011

(54) SPIRAL LEAD

(75) Inventor: Kevin K. Tidemand, East Bethel, MN (US)

Correspondence Address: MEDTRONIC, INC., 710 MEDTRONIC PARKWAY NE, MINNEAPOLIS, MN, 55432-9924, US

(73) Assignee: MEDRONIC, INC., MINNEAPOLIS, MN (US)

(21) Appl. No.: 12/937,571

(22) PCT Filed: Jan. 23, 2009
(86) PCT No.: PCT/US09/31787

§ 371 Date: Oct. 13, 2010

(30) Foreign Application Priority Data
Apr. 21, 2008 (US) 61/046499

(51) Int. Cl. A61N 1/00

(52) U.S. Cl. 607/116

(57) ABSTRACT

An implantable medical lead includes a lead body having a proximal end, a distal end, and a spiral segment between the proximal end and the distal end. The spiral segment has a center, a proximal beginning point and a distal ending point. The proximal beginning point is closer to the center than the distal ending point. The lead further includes a contact element disposed in proximity to the proximal end of the lead body and an array of electrodes disposed at the spiral shaped segment of the lead body. A conductor extends within the lead body from the contact element to an electrode of the array and electrically couples the contact element and the electrode of the array.

### FIELD

This application relates to medical devices, more particularly to implantable leads for delivering electrical signals.

### BACKGROUND

Implantable electrical signal generators, such as neurostimulators, have been used to treat a variety of diseases. Such devices generate electrical signals that are transferred to a patient's tissue through electrodes disposed on a distal end portion of a lead. The proximal end portion of a lead typically contains a number of connector rings corresponding to the number of electrodes. Conductors run within and along the lead body and electrically couple the connector rings to the electrodes. The proximal end portion of the lead is inserted into a connector region of a signal generator such that electrical contact is made between discrete contacts in the connector portion and the connector rings of the lead. Thus, electrical signals generated by the signal generator may be delivered to a patient's tissue via the electrodes.

Many leads contain a plurality of electrodes. One reason for employing a plurality of electrodes is to allow flexibility for an electrical signal to be delivered to an appropriate tissue location of the patient. For example, if the distal portion of the lead containing the electrodes moves over time, the signal generator may be instructed to deliver an appropriate to different electrodes to compensate for the movement. In addition, having a number of electrodes on a lead can allow for some variability in surgical placement. Once the lead is implanted, various electrodes, or combinations thereof, may be tested until a desired effect is obtained.

One example of a lead for use in cortical stimulation is a disc shaped paddle lead having a surface configured to be placed adjacent a patient's brain. An array of electrodes are placed on or exposed through that surface. The area of the surface and the number of electrodes in the array are designed to allow for, among other things, the ability to select appropriate electrodes or electrode combinations to provide a therapeutic effect.

### BRIEF SUMMARY

Leads having spiral shaped segments that can provide an array of electrodes over a surface area similar to disc shaped paddle leads are described herein. The spiral shaped leads described herein can be inserted into burr holes in a patient's skull and may allow for a smaller opening than would be required for similarly-sized disc-shaped (or other-shaped) paddle leads providing a similar surface area of electrode coverage.

In an exemplary embodiment, an implantable medical lead includes a lead body having a proximal end, a distal end, and a spiral segment between the proximal end and the distal end. The spiral segment has a center, a proximal beginning point and a distal ending point. The proximal beginning point is closer to the center than the distal ending point. The lead further includes a contact element disposed in proximity to the proximal end of the lead body and an array of electrodes disposed at the spiral shaped segment of the lead body. A conductor extends within the lead body from the contact element to an electrode of the array and electrically couples the contact element and the electrode of the array.

In an exemplary embodiment, a method includes creating an opening extending through a skull of a patient and inserting a distal end of a lead into the opening. The lead has a spiral portion extending from the distal end to a proximal portion of the lead. The proximal portion is closer to the center of the spiral portion that the proximal portion of the spiral portion. The method further includes turning the lead to advance at least a portion of the spiral portion of the lead into the opening to position at least a portion of the spiral portion of the lead adjacent the patient's brain.

By having an electrode array disposed along a spiral segment of a lead, as opposed to a disc shaped paddle, an electrode array covering a similar surface area may be placed adjacent the cortex of a patient without requiring drilling of a large burr hole. By having the distal ending point of the spiral segment further from the center of the spiral segment than the proximal beginning point, the spiral segment may be readily introduced through the burr hole. These and other advantages will be readily understood from the following detailed descriptions when read in conjunction with the accompanying drawings.

### BRIEF DESCRIPTION OF THE DRAWINGS

FIG. 1 is a schematic diagrammatic representation of a representative electrical signal generator system implanted in a patient.

FIG. 2 is a schematic perspective view of a disc shaped paddle lead.

FIG. 3 is a schematic perspective view of an exemplary embodiment of a lead having a spiral shaped segment.

FIG. 4 is a schematic bottom view of an exemplary embodiment of a spiral shaped segment of a lead.

FIG. 5 is a schematic perspective view of an exemplary embodiment of a lead having a substantially planar spiral shaped segment.

FIG. 6A is a schematic diagrammatic representation of a perspective view of an exemplary embodiment of a lead having a spiral shaped segment and a cross-section of a skull.

FIG. 6B is a schematic cross-section of a distal portion of an exemplary embodiment of a lead having a spiral segment being inserted into a burr hole in a skull.

FIG. 6C is a schematic perspective view of an exemplary embodiment of a spiral segment of a lead inserted through a burr hole in a skull, with a portion of the skull being cut away.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “proximal” and “distal” refer to position relative to an implantable electrical signal generator. For example, a proximal portion of a lead is a portion nearer a signal generator, and a distal portion is a portion further from the signal generator.

As used herein, “signal generator” and “pulse generator” are used interchangeably. It will be understood that a pulse generator may generate an electrical signal or a plurality of electrical signals that are not pulses.

This disclosure relates to implantable medical leads having a spiral segment. The spiral segment has a distal ending point that is further from the center than the proximal beginning point. An array of electrodes is disposed at the spiral segment. The array may be regular or irregular. The electrodes of the array may be selected for applying electrical stimulation signals to a surface area of tissue that may be similar to that achievable by disc shaped paddle leads. However, unlike disc shaped paddle leads, the spiral electrodes described herein can be introduced through a small opening in the patient, such as a burr hole in the patient's skull. The leads described herein may be employed for any suitable purpose and in conjunction with any suitable active electrical implantable device.

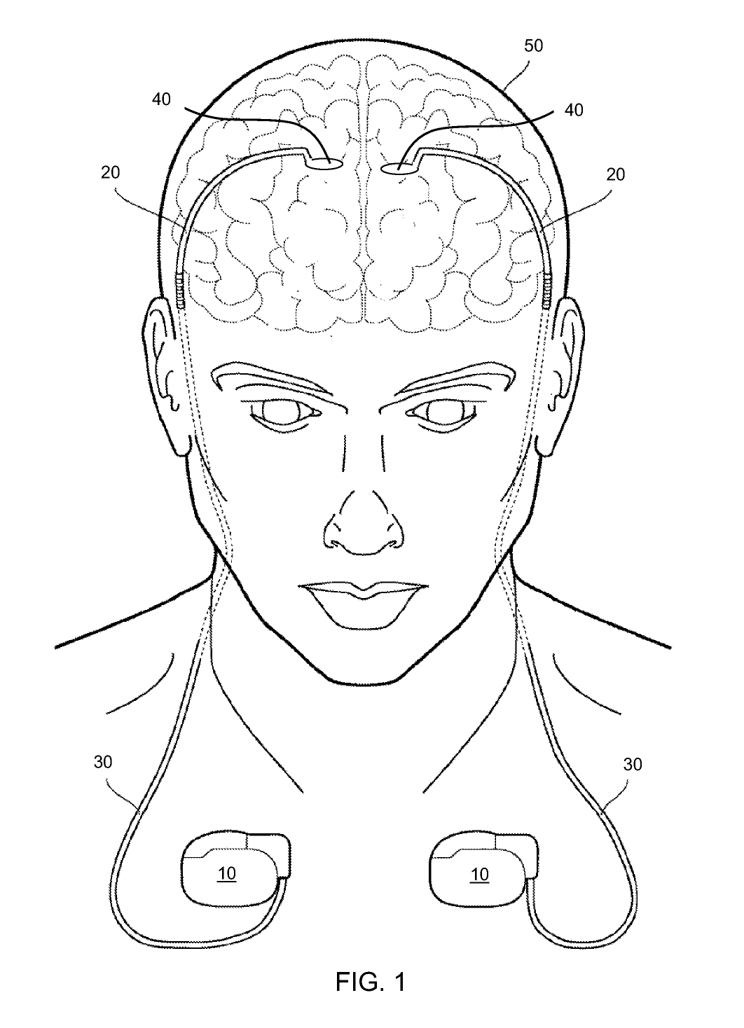

Referring to FIG. 1, a schematic diagrammatic representation of an electrical signal generator system implanted in a patient for application of electrical signals to the patient's cortex. Implantable pulse generators 10 are shown implanted in a pectoral region of the patient. However, it will be understood that pulse generators 10 may be implanted at any medically acceptable location of the patient. A lead extension 30 is operably coupled to the pulse generator 10. A lead 20 is operably coupled to the pulse generator 10 via extension 30. Lead 20, in various embodiments, is directly coupled to pulse generator 10 without use of an extension 30. A distal portion 40 of lead 20 is shown implanted and positioned for application of electrical signals to the cortex of the patient. While two electrical signal generators 10 are shown implanted in the patient, it will be understood that one signal generator 10 having connections for two extensions 30 or leads 20 may be used.

To place distal portion 40 of lead 20 adjacent to the cortex, a burr hole (not shown) is drilled in the patient's skull 50. The diametric dimension of the burr hole is sufficiently large to allow distal portion 40 of lead 20 to pass through the burr hole. The proximal end of the lead is typically tunneled between the patients scalp and skull 50 to connect to extension 30, which is tunneled subcutaneously to connect with pulse generator 10. Electrical signal generator is capable of generating electrical signals that may be applied to tissue of the patient, such as the cortex, for diagnostic or therapeutic purposes. Pulse generator 10 typically includes a power source and electronics for sending electrical signals to the cortex via the distal portion 40 of lead 20. Implantable pulse generator 10 may receive instructions via telemetry from a programmer (not shown) located external to the patient, such as a physician or patient programmer device.

While the signal generator 10 depicted in FIG. 1 and as discussed below is configured for cortical stimulation, it will be understood that the leads having spiral segments described herein may be used with any active electrical device, such as a cochlear implant; a sensing device; a signal generator such as a cardiac pacemaker or defibrillator, an other neurostimulator (such as a spinal cord stimulator, a brain or deep brain stimulator, a peripheral nerve stimulator, a vagal nerve stimulator, an occipital nerve stimulator, a subcutaneous stimulator, etc.), a gastric stimulator; or the like.

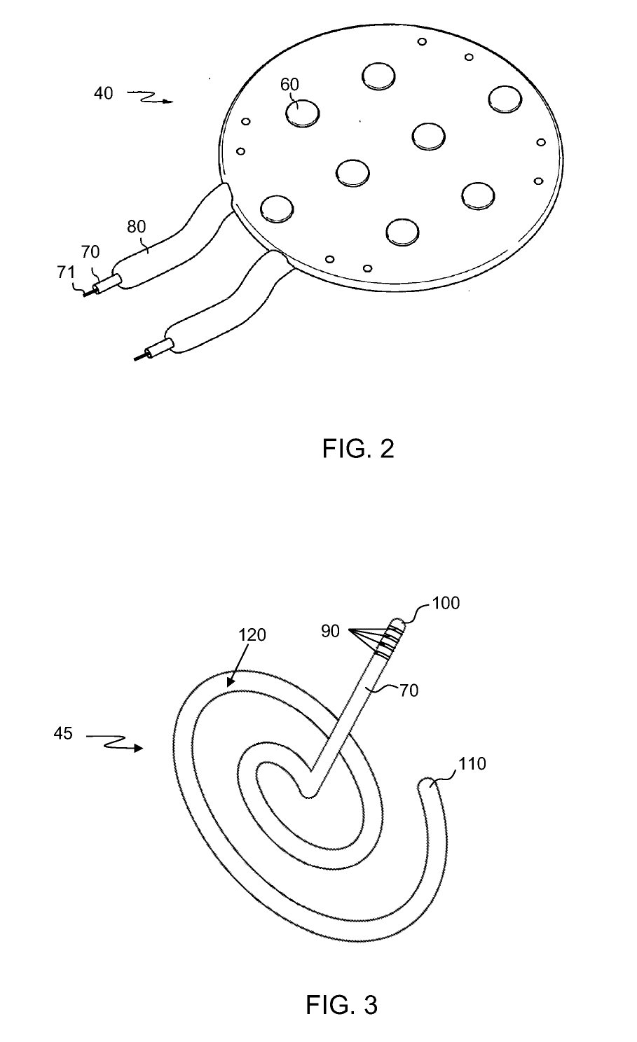

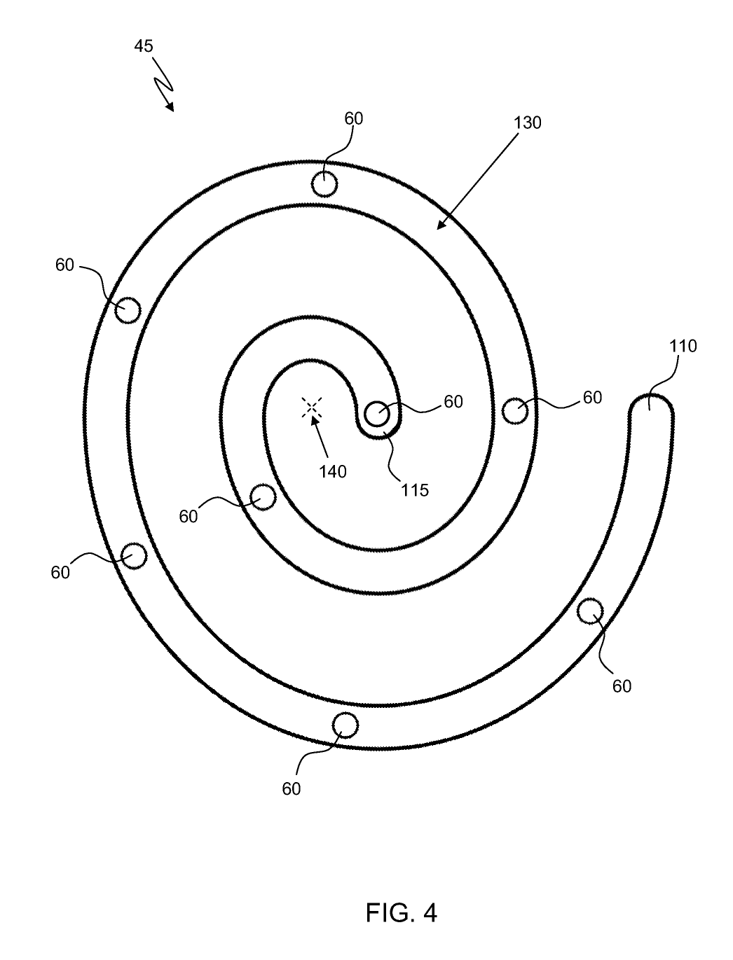

Referring now to FIG. 2, a schematic perspective view of a disc shaped paddle lead is shown. The lead includes a lead body 70 and a paddle shaped distal portion 40 including an array of electrodes 60. One or more conductors 71 run within the lead body 70 and operably couple the electrodes 60 to contacts at the proximal portion of the lead (not shown) The electrodes 60 are distributed along a surface of the disc shaped paddle portion 40 that is configured to be placed adjacent and face the cortex. Once in place, different electrodes or combinations of electrodes may be employed to achieve a desired effect. The disc shaped paddle lead may be a lead as described in, e.g., US 2004/0243205, entitled “IMPLANTABLE CORTICAL NEURAL LEAD AND METHOD”, published Dec. 2, 2004. The depicted lead includes strain reliefs 80. The disc shaped paddle lead in FIG. 2 is shown for purposes of comparison to the leads having spiral segments shown in FIGS. 3-6.

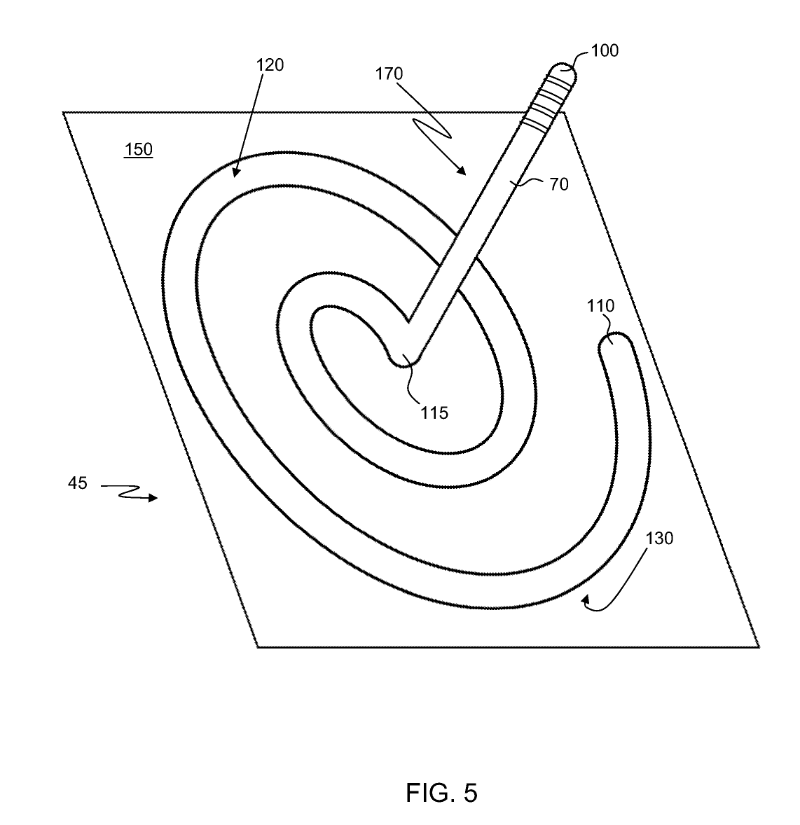

Referring now to FIGS. 3-4, representative exemplary leads having spiral segments 45 are shown. In FIG. 3 a perspective view is shown. In FIG. 4 a bottom view is shown. The lead has a lead body 70 having a proximal end 100 and a distal end 110 and the spiral segment 45 between the proximal 100 and distal 110 end. In the depicted embodiments, the spiral segment 45 includes the distal end 110 of the lead body 70. The spiral segment has a top 120 and bottom 130. One or more contact elements 90, such as contact rings, are disposed in proximity to proximal end 100 of lead body 70. The contact elements 90 are disposed in, on, or about the lead body 70 such that the contact elements 90 may be electrically coupled to an active medical device, such as an electrical signal generator, or a lead extension or other adaptor between active device and the lead. Conductors (not shown) extend within the lead body and electrically couple the contact elements 90 to electrodes 60 on, in, or exposed through (generally disposed “at”) bottom of the spiral segment 40. Typically each electrode 60 of the array of electrodes is electrically coupled to a discrete contact element 90.

Any number of electrodes 90 may be disposed at the bottom 130 of the spiral segment 45. For example, four, five, six, seven, eight, sixteen, thirty-two or sixty-four electrodes 60 may be disposed at bottom 130 of spiral segment 45. In some embodiments, the area of tissue that electrodes 60 of spiral segment 45 may contact or cover is similar to the area that may be covered by a disc-shaped paddle lead.

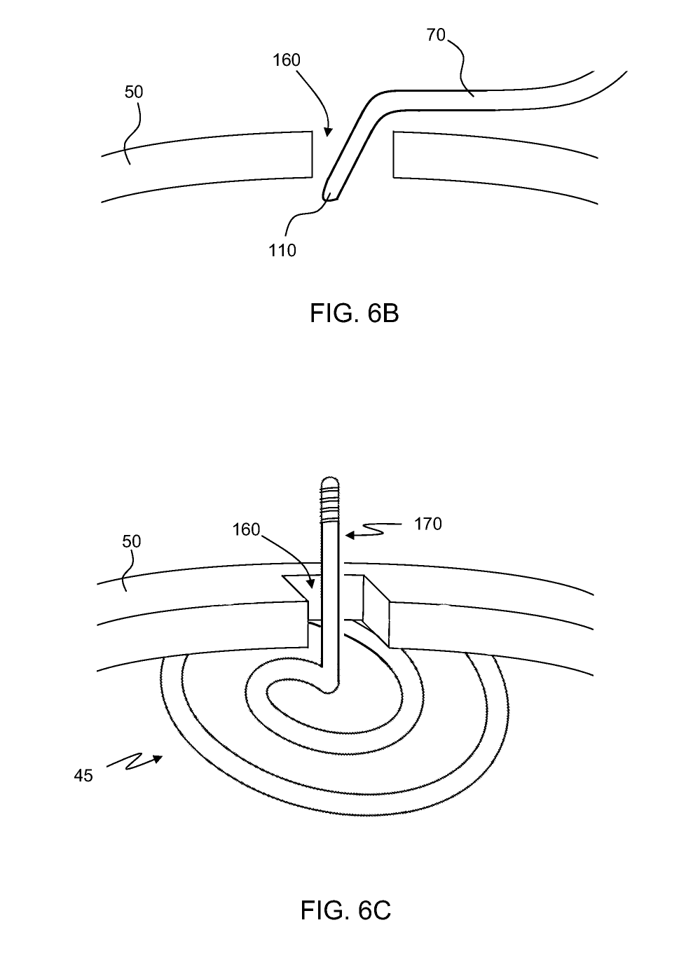

The spiral segment 45 has a center 140. As used herein, “center” in the context of a spiral segment 45 means within an area defined by the innermost turn of the spiral. If the spiral segment 45 is substantially planar, the center 45 may be a point within the plane that is within the innermost turn of the spiral. However, the center is not limited to being an exact geometric center and the precise location is not essential. If the spiral segment 45 forms a three-dimensional spiral, the center 45 may be an axis line running through the tightest turn of the spiral (in addition to the other turns of the spiral). As shown in FIG. 4, the spiral segment 45 has a proximal beginning point 115, which is the proximal most point on the lead body 70 where the spiral of the spiral segment 45 begins. The spiral segment 45 also has a distal ending point, which in FIG. 4 is the distal end 110 of the lead body. The distal ending point is the distal most point of the lead body that forms part of the spiral of the spiral segment. As can be seen in, e.g., FIG. 4 the proximal beginning point 115 of the spiral segment 45 is closer to the center 140 than the distal ending point, which is the distal end 110 in FIG. 4. As will be discussed further with regard to FIG. 6, such a spiral arrangement where the proximal beginning point is closer to the center than the distal ending point facilitates insertion of the spiral segment 45 into an opening or orifice. In various embodiments, the proximal beginning portion 115 of the spiral segment 45 at the center 140 of the spiral segment.

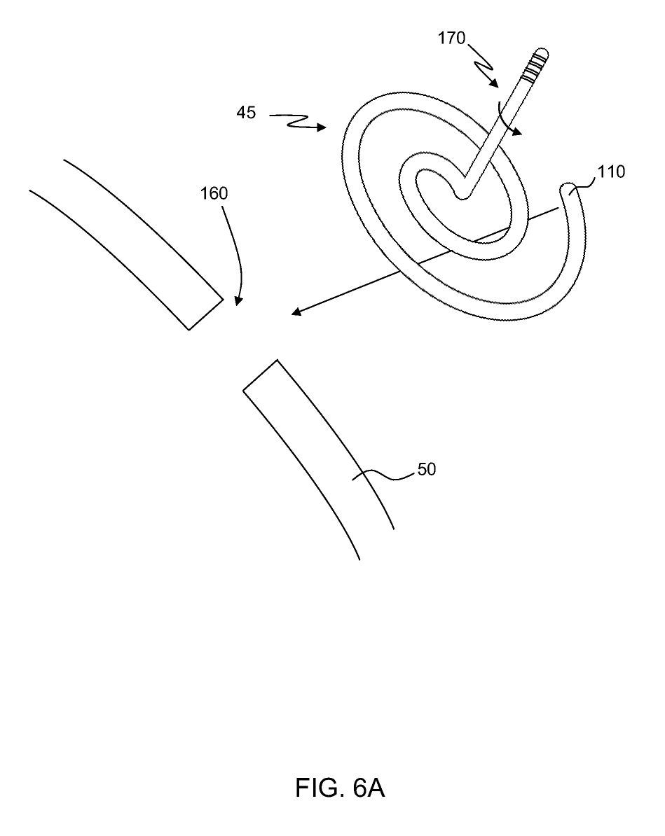

As shown in FIG. 5, spiral segment 45 may be substantially planar. The bottom 130 of spiral segment 45 may lie substantially within a plane 150 in its relaxed state, allowing the bottom 130 to engage tissue of a patient, such as the patient's cortex, when implanted in the patient. The electrodes (not shown in FIG. 5) are positioned such that the electrodes may be in electrical communication with the tissue when the bottom 130 of the spiral segment 45 engages the tissue. In various embodiments, the electrodes face the tissue with substantially the same orientation. Of course, the orientation of the electrodes of the array may be varied if desired.

As shown in FIG. 5, the lead body 70 may further include a substantially linear segment 170 between the proximal end 100 of the lead body 70 and the proximal beginning point 115 of the spiral segment 45. The substantially linear segment 170 extends out of the plane 150 and away from the top 120 (in the direction from bottom to top) of the spiral segment 45. In various embodiments, the linear segment 170 is substantially normal to the plane 150. In many embodiments where spiral segment 45 is three dimensional (not depicted), the linear segment 170 extends away from the top 120 of the spiral segment 45.

While spiral segment may take any shape, in various embodiments spiral segment is flat like a paddle lead. The spiral segment may be generally circular, oblong, or any other desired shape, whether in relatively flat or three-dimensional.

Leads having spiral segments as described herein may be made according to any known or future developed process. For example, the body material of devices may be injection molded or extruded. In some situations it may be desirable to reflow body material from thermoplastic polymers. Body material is typically made of polymeric material, such as polyurethane, polycarbonate, or silicone or combinations thereof. Body material typically has an elastic modulus of less than 15 ksi (less than 100 MPa), e.g. between 0.5 and 5 ksi (between 3.5 and 35 MPa).

A reinforcement member may be incorporated into body material to provide additional strength or to increase stiffness of, for example, the spiral segment. A reinforcement member may be extruded, molded, or the like. A reinforcement member may be made of metallic material or of non-conductive material. Exemplary non-conductive materials for use as reinforcement member include polyester polymeric materials, such as polyethylene napthalate, polyethylene terephthalate, polyether ether ketone, polyetherether ketone or the like.

Electrodes may be formed of electrically conductive biocompatible materials, such as platinum or platinum iridium. Contacts and conductors may be formed of electrically conductive biocompatible materials, such as platinum, platinum iridium, titanium, tantalum, nickel-cobalt-chromium-molybdenum alloys, or the like. Conductors may comprise braided strand wire.

One non-limiting way to make a spiral segment of a lead as described herein is to cut a spiral slit in a circular paddle. The electrodes (and conductors) may be placed in a spiral pattern to allow for such a cut. Of course, any suitable method for making a lead may be employed or modified to make a lead having a spiral segment as described herein.

Referring now to FIGS. 6A-C, a diagrammatic depiction of an overview of an insertion of a spiral segment 45 of a lead into an opening or burr hole 160 created in a skull 50 of a patient is shown. The distal end 110 of the lead is inserted into the opening 160. In the depicted embodiment, the distal end 110 is the distal ending point of the spiral of the spiral segment 45 of the lead. Once the distal end 110 of the lead is inserted into the opening 160. The lead may be turned to advance the spiral segment 45 through the burr hole 160, allowing the bottom surface of the spiral segment (and thus the electrodes) to be positioned adjacent and facing the patient's brain, particularly the cortex. In various embodiments, the lead may be turned by applying a turning force to the substantially linear portion 170 of the lead.

As can be seen from the drawings presented in FIGS. 6A-C, when the lead having a spiral segment 45 is configured to be advanced through a burr hole 160 of a skull, it is desirable, but not essential, that the spiral segment 45 be sufficiently flexible to bend for placement in the hole 160 (see, e.g., FIG. 6B), yet be sufficiently stiff to retain the spiral shape as it is being advanced through the hole and positioned adjacent the cortex (see, e.g., FIG. 6C). Materials having sufficient buckling stiffness to permit advancing the spiral segment 45 without collapsing, having sufficient lateral stiffness to permit retaining the shape of the spiral segment 45 while advancing the lead, and having sufficient flexibility in the plane of the spiral segment 45 to conform to the anatomical shape of the surface of the brain may be readily selected by those of skill in the art upon an appreciation of the teaching herein.

Referring to FIGS. 6A-C, a lead having a spiral segment 45 may be inserted into a burr hole having a small diametric dimension relative to a disc shaped paddle lead. Further evident, is that having the distal ending point 110 of the spiral segment 45 further from the center of the spiral than the proximal beginning point serves to facilitate insertion through the burr hole 160. Such a configuration allows for ease of screwing or advancing the spiral segment 45 into the hole 160 around a central axis, similar to a typical corkscrew. This results in a proximal section that is centrally located in the center of the burr hole and in the centroid of the electrode array.

When the substantially linear portion 170 extends from the center of the spiral segment 45, the spiral segment 45 may be inserted into and through the burr hole 160 such that the spiral segment 45 is substantially equally distributed under the skull 50 around the burr hole 160. In some instances, it may be desirable to have the substantially linear portion 170 extend from the spiral segment 45 at a position off-center to allow for insertion of the spiral segment 45 off-center of the burr hole 160. Some degree of steerability may be imparted on the lead. Also, in some embodiments, it may be desirable for the linear portion 170 to extend away from the spiral segment 45 at angle other than normal for purposes of steering.

While not shown, it will be understood that the linear portion 170 may include a highly flexible section that can be run along the outer surface of the patient's skull 50.

Thus, exemplary embodiments of the spiral lead are disclosed. One skilled in the art will appreciate that the present disclosure can be practiced with embodiments other than those disclosed. The disclosed embodiments are presented for purposes of illustration and not limitation, and the present invention is limited only by the claims that follow.

1-20. (canceled)

21. An implantable medical lead comprising:
a lead body having a
(i) proximal end,
(ii) a distal end,
(iii) a spiral segment between the proximal end and the distal end, the spiral segment having a center, a proximal beginning point and a distal ending point, wherein the proximal beginning point is closer to the center than the distal ending point, wherein the spiral segment of the lead body has a top and bottom and is substantially planar, and
(iv) a substantially linear segment between the proximal end of the lead body and the proximal beginning point of the spiral segment, wherein the substantially linear segment extends out of the plane away from the top of the spiral segment;
a contact element disposed in proximity to the proximal end of the lead body; and
an array of electrodes disposed at the spiral shaped segment of the lead body, wherein the electrodes of the array are electrically coupled to the contact element, the bottom of the spiral segment is configured to engage tissue of the patient, and the electrodes of the array are positioned such that the electrodes are in electrical communication with the tissue when the bottom of the spiral segment engages tissue.

22. An implantable medical lead according to claim 21, wherein the substantially linear segment extends substantially normal to the plane.

23. An implantable medical lead according to claim 21, wherein the substantially linear segment extends from the center of the spiral segment.

24. A system comprising:
an implantable medical lead according to claim 21; and
an electrical signal generator operably couplable to the lead such that a signal generated by the generator is deliverable via one or more electrodes of the array.

25. A method for manufacturing a lead comprising:
forming a lead body having
(i) a proximal end,
(ii) a distal end,
(iii) a spiral segment between the proximal end and the distal end, the spiral segment having a center, a proximal beginning point and a distal ending point, wherein the proximal beginning point is closer to the center than the distal ending point, wherein the spiral segment of the lead body has a top and bottom and is substantially planar, and
(iv) a substantially linear segment between the proximal end of the lead body and the proximal beginning point of the of spiral segment, wherein the substantially linear segment extends out of the plane away from the top of the spiral segment;
disposing an array of electrodes at the spiral shaped segment of the lead body such that the electrodes are exposed through the bottom of the spiral segment;
disposing a contact element in proximity to the proximal end of the lead body; and
electrically coupling the contact element to an electrode of the array.
